(19) United States
(12) Patent Application Publication
(10) Pub. No.: US 2019/0069511 A1
(43) Pub. Date: Mar. 7, 2019

(54) Weight Management System for Pets, Associated Method, and Device

(71) Applicant: Nestec S.A., Vevey (CH)

(72) Inventors: Youcef AIT BOUZIAD, Echandens (CH); Cédric DUBOIS, Lavigny (CH); Jean-Enmanuel SERRE, Le Mesnil-Eudes (FR); Catherine THERON, Sains en Amienois (FR); Praveen Kumar VADI, Aurora, OH (US)

(21) Appl. No.: 16/085,226

(22) PCT Filed: Mar. 27, 2017
(86) PCT No.: PCT/EP17/57159

§ 371 Date: Sep. 14, 2018

(30) Foreign Application Priority Data
Apr. 6, 2016 (EP) 16163961.2

(51) Int. Cl. A01K 5/02

(52) U.S. Cl. CPC A01K 5/0283; A01K 5/0291

(57) ABSTRACT

The invention relates to a pet weight management system (100) comprising:
a controller (10) receiving data from a pet, these data comprising at least one or more invariant parameters (11) and one or more variable parameters (12), the controller (10) establishing a nutritional program (31) as a function of these data, the nutritional program (31) comprising at least one or a plurality of feeding cycles, each feeding cycle comprising at least a caloric reduction period and at least a caloric maintenance period;
a nutrition management device (20) operatively linked to the controller (10) receiving the information on the nutritional program (31) to follow, and dosing and/or dispensing via weighing or volume measurement;
a composition kit (30) comprising the required nutritional products in the required quantities needed to follow the nutritional program (31) established.

The invention further relates to a nutrition management device (20) used in a system (100) as the one described, comprising a weighing device further configured to accommodate at least part of the nutritional products to be administered to the pet. Moreover, the invention relates to a composition kit (30) to be used in a system (100) as described and to a method for promoting weight loss in a pet using such a system (100).

### CROSS REFERENCE TO RELATED APPLICATIONS/INCORPORATION BY REFERENCE STATEMENT

This application is a US national stage application filed under 35 USC § 371 of International Application No. PCT/EP2017/057159, filed Mar. 27, 2017; which claims priority to EP App No. 16163961.2, filed Apr. 6, 2016. The entire contents of the above-referenced patent applications are hereby expressly incorporated herein by reference.

### TECHNICAL FIELD

The present disclosure is directed to a weight management system for pets, as well as to an associated method for using such a weight management system. Furthermore, the present disclosure is directed to a nutrition management device used in such a system and applying the mentioned method.

### BACKGROUND

Pets are becoming more frequent in houses in the last years: however, due to working timings and also to frequent travelling of most of the family members, it is not easy to maintain a proper and balanced diet and nutritional feeding for your pet. Moreover, pets are becoming more sedentary and usually ingest more than what they consume, which makes overweight a frequent problem for pets at present.

When managing an animal's weight, particularly promoting weight loss, it is a very common practice to reduce caloric intake by restricting the amount of food available to the animal on a chronic and daily basis. Generally, restricting daily food intake chronically causes the animal to be hungry, which triggers undesired behaviors. In addition, managing weight loss by chronic and daily caloric reduction may cause undesirable results that affect body composition. Animals that lose weight often lose both fat body mass and lean body mass and reduce their daily energy expenditure. Reduced daily energy expenditure caused by chronic daily caloric reduction during weight loss makes animals to regain their lost weight easily and makes it very difficult, for the animals to maintain their ideal body weight after weight loss. Therefore, it is frequently more desirable when losing weight to lose excess fat body mass while preserving as ranch lean body mass as possible and maintaining higher daily energy expenditure.

Document WO2014/031836 A1 belonging to the same applicant describes a method for promoting weight loss by an animal, comprising a caloric maintenance and a caloric reduction period. One non-limiting object of the present disclosure is to provide a weight management system and an associated method to apply in an automatic and convenient way the method for promoting weight loss described in the previously mentioned document. Moreover, another non-limiting object of the present disclosure is to provide a nutrition management device applying such a method of promoting weight loss.

It is known in the state of the art, for example as per document WO 02/085140, a body weight management system for humans and also for domestic animals, the system comprising diet tracking systems, devices to estimate the energy expenditure, compositions with satiety agents and a weight manager providing information, feedback or combinations thereof to the individual. By controlling these parameters, the gain, lose or weight maintenance can be determined. The diet tracking system can be software loaded in a hand held programmable device or in a computing device. The problem of these known systems is that they are complicated, require further number of elements and are not easy to handle or use. Moreover, they are mostly directed to humans and not particularly to pets.

Also known from document US 2013/0121461 A1 is a method of managing weight in a companion animal determining the actual body percentage of body fat or lean body mass in the animal, then applying a regression analysis deriving one or more equations to predict this value in time so as to apply an effective weight loss regimen for the animal. A kit comprising means for communicating information or instructions for such a method is also described in this document. However, no convenient or easy system is described in this document in order to apply such a method.

Accordingly, there is the need to provide a weight management system for pets which is easy and convenient and which overcomes the drawbacks and disadvantages in the known prior art as previously presented.

### BRIEF DESCRIPTION OF THE DRAWINGS

Further features, advantages, and objects of the present disclosure will become apparent for a skilled person when reading the following detailed description of embodiments of the present disclosure, when taken in conjunction with the figures of the enclosed drawings.

FIG. 1 shows a general overview of the weight management system for pets according to the present disclosure.

FIG. 2 shows a general overview of the method for using a weight management system according to the present disclosure, as presented in FIG. 1.

FIGS. 3a and 3b show details of the nutrition management device used in a weight management system for pets according to the present disclosure.

### DETAILED DESCRIPTION

According to a first aspect, the present disclosure refers to a pet weight management system comprising: a controller receiving data from a pet, these data comprising at least one or more invariant parameters and one or more variable parameters, the controller establishing a nutritional program as a function of these data, the nutritional program comprising at least one or a plurality of feeding cycles, each feeding cycle comprising at least a caloric reduction period and at least a caloric maintenance period; a nutrition management device operatively linked to the controller receiving the information on the nutritional program to follow, and dosing and/or dispensing via weighing or volume measurement; a composition kit comprising the required nutritional products in the required quantities needed to follow the nutritional program established.

Typically, the caloric reduction period and the caloric maintenance period are intermittent periods of the same duration. In certain non-limiting embodiments, the caloric reduction period contains from 5% to 90% fewer calories than the caloric maintenance period.

In certain non-limiting embodiments of the system of the present disclosure, the invariant parameters are at least one or a plurality of: breed, age, gender, activity level, behavior and/or sterilization. Also, in a particular (but non-limiting) embodiment, the variable parameters are at least the pet's weight.

The nutritional program typically comprises at least one or a plurality of the following data: type of nutritional product or composition, quantity of the nutritional product or composition, total duration and/or administration frequencies of the nutritional program.

According to a second aspect, the present disclosure further relates to a nutrition management device used in a system as described, comprising a weighing device further configured to accommodate at least part of the nutritional products to be administered to the pet.

In certain non-limiting embodiments, the nutrition management device is configured as a dosing device or the like.

According to the present disclosure, the nutrition management device, in certain non-limiting embodiments, further comprises a wireless connection mode allowing its connection to the controller in the system, such as by Bluetooth or NFC or W-Fi.

Typically, the nutrition management device is configured to synchronize data automatically each time it is near the controller.

The nutrition management device of the present disclosure typically comprises a screen where the information on the nutritional program to follow is graphically represented.

Typically, the nutrition management device further comprises at least one or more operating buttons configured to adjust one or more parameters from the nutritional program.

According to a third aspect, the present disclosure relates to a composition kit used in a system as the one described, adapted to follow a certain nutritional program.

Typically, the composition kit comprises at least one or more nutritional products that can be administered to follow one or more nutritional programs.

According to the present disclosure, the composition kit can further comprise at least one or a plurality of weight loss agents taken from the list of: carnitine, isoflavones, pyruvate, fish oil, DMA, EPA, fibers, calcium, resistant starch, medium chain triglycerides, green tea extract, phentemine, diethylpropion, orlistat, sibutramine, megestrol phenylpropanolamine, oxaudrolone, oleoylethanolamide, probioties that promote weight loss, ephedra, conjugated imoleic acid, glucomannan, or the like.

Yet according to a fourth aspect, the present disclosure refers to a method for promoting weight loss in a pet, using a system as the one described. This method comprises: establishing a certain nutritional program as a function of one or more invariant parameters and one or more variable parameters, the nutritional program comprising at least one or a plurality of feeding cycles, each feeding cycle comprising at least a caloric reduction period and at least a caloric maintenance period; updating the nutritional program established in a nutrition management device; following the nutritional program through the device, operatively linked to the controller.

In certain non-limiting embodiments, in the method of the present disclosure, a new updated nutritional program is established each time at least one of the variable parameters changes.

Typically, the caloric reduction period and the caloric maintenance period in the method according to the present disclosure are intermittent periods of the same duration.

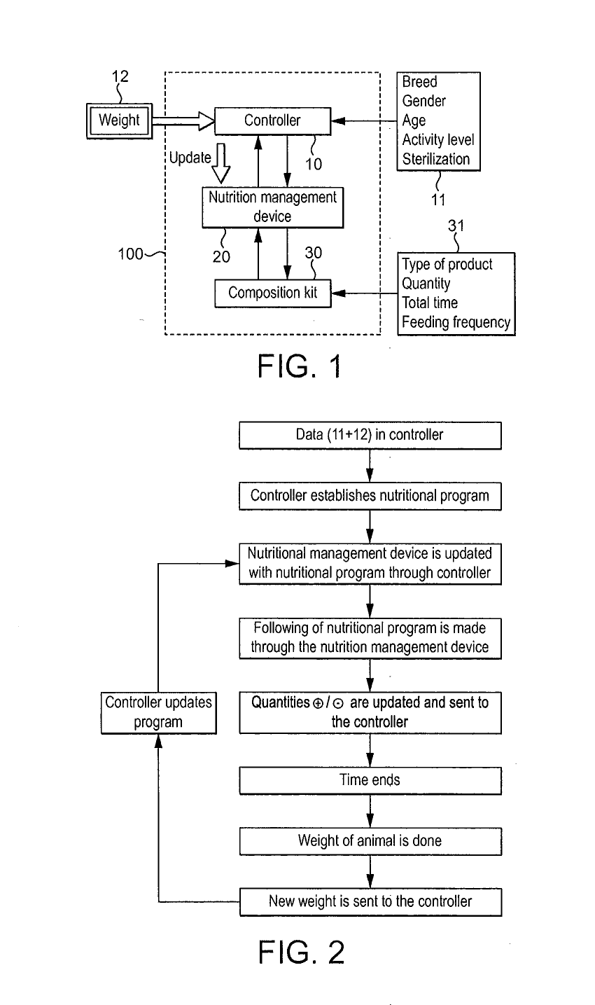

Referring now to FIG. 1, a weight management system 100 for pets according to the present disclosure is presented. The system of the present disclosure comprises a controller 10, a nutrition management device 20 and a composition kit 30. The controller 10 is provided with two types of entry parameters: invariant parameters 11 depending on the pet being monitored and variable parameters 12. The invariant parameters 11 are at least one or a combination of: breed, gender, age, activity level or behavior (if the pet has a low, medium or high activity level, and/or if the pet is more an indoor pet or an outdoor pet which is directly related to a certain activity level) and/or sterilization (i.e. if the pet has been sterilized or not). The variable parameters 12 are typically the weight of the pet, this being the parameter used for updating a nutritional program 31 established for the pet.

By pet it is to be understood any kind of companion animal meaning domesticated animals such as dog, cats. Rabbits, guinea pigs, ferrets, hamsters, mice, gerbils, horses, cows, goats, sheep, donkeys, pigs and the like.

The controller 10, once provided with the entry or input parameters 11 and 12 is able to establish a nutritional program 31 to be followed by the pet in order for it to lose weight. The nutritional program 31 comprises, as shown in FIG. 1, one or a plurality of data, such as for example: the type of product or composition to be given to the pet, the quantity of this product to be provided, and both of these (type and quantity) to be provided at a certain feeding frequency, to be established over a certain period of total time; the type and quantity of product can also vary for each frequency period. The controller 10 then sends the calculated nutritional program 31 to the nutrition management device 20. The device 20 then uses a certain composition kit 30 comprising the necessary ingredients (products of certain types and having certain quantities) for following a certain nutritional program 31 for a certain period of time.

As represented in FIG. 1, each time the weight of the pet (typical variable parameter 12) changes, the system is typically updated so a new nutritional program 31 is established, taking into consideration the present weight of the pet, therefore a different composition kit 30 shall be used.

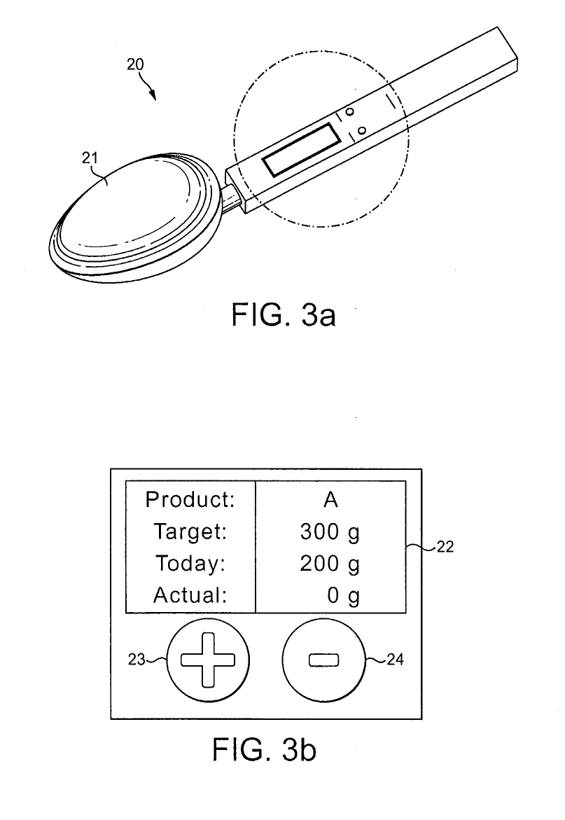

Referring now to FIG. 2, the method according to the present disclosure used in a weight management system 100 is presented. Once both invariable data 11 and variable parameters 12 are introduced in the controller 10, it establishes a certain nutritional program 31 based on these, this specific nutritional program being then sent to the nutrition management device 20. The follow-up of this program 31 is effected by means of the device 20, typically informing the user on the quantity and type of product to be given to the pet in a certain frequency. At the end of the total period of time the nutritional program is established for, an alert appears in the device 20 asking the user to weight the pet once again (typical only variable in the system of the present disclosure) so a new updated and adequate nutritional program 31 is—established. The method can be applied a plurality of times, until a desired weight is achieved: this desired or ideal weight is typically known by the controller 10, once the invariant parameters 11 are established. The control of the quantities given or withdrawn to the pet is also done by means of certain functionalities in the device 20, as it will be further explained in detail in what follows.

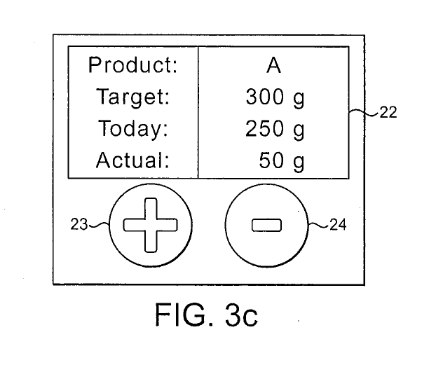

FIGS. 3a and 3b show a particular (but non-limiting) execution of a nutrition management device 20 according to the present disclosure, used to follow the nutritional management method previously described.

The device 20 is typically shaped as a dosing device, such as (but not limited to) as a spoon or the like, as shown in FIG. 3a: the spoon is programmed and charged with the information on the nutritional program 31 to follow once it is connected to the controller 10. The device 20 further updates its nutritional program 31 each time the weight of the pet (typical variable parameter 12) changes.

The device 20 comprises a wireless connection mode, typically Bluetooth or NFC or W-Fi; the controller 10 is typically a smartphone, a router or a PC. The data on the nutritional program 31 can be programmed directly in factory (not at user level) or can be downloaded via web. The device 20 can synchronise data automatically each time it is near the server 10, for example, or it can be synchronised via a dedicated button. Furthermore, the device 20 comprises an embedded memory (typically lasting up to one month) allowing the device to work without synchronisation up to one month.

The device 20 typically comprises a weighing part 21, further working as a dosing part, configured as a concave spoon or container allowing to hold the product that will be given to the pet and weighing it at the same time and also allowing to dose this product to the container from where the pet eats. The device 20 further comprises a screen 22 and operating buttons, addition and subtraction buttons, 23 and 24, respectively. The screen 22 typically shows the user the following information: the type of product that has to be provided to the pet, the target total quantity of the product to be provided in a certain period of time, the quantity of that product that has been provided in a certain frequency time and the weighed quantity in that moment. The addition button 23 allows to adjust the quantity over a certain threshold, while the subtraction button 24 allows to diminish or lower the quantity over a certain threshold value. The subtraction button 24 also provides the possibility of storing in the device memory the information on certain quantity of product that has been removed from the total amount given, for example because the pet did not eat it and we want to throw it away or because we made a mistake and we gave too much quantity of food product and we have to subtract it from the total. The weighing part 21 works dosing by weighing and/or by volume measurement of the quantity of product placed in it.

As shown in FIG. 3a, the nutrition management device 20 of the present disclosure is typically configured as a spoon or the like, comprising a weighing part 21 which is also used as dosing part. The screen 22 of the device 20 typically shows the following information, as represented in FIG. 3b: type of product that has to be administered to the pet (for example, as shown in FIG. 3b, product of type A) and the target (daily target quantity to be administered to the pet; the quantities are always measured and determined daily as it is this the feeding cycle for pets). The two lower quantities vary according to what has been already given (accumulated) to the pet that day and according to what is being weighed in the weighing part 21 in that specific moment. For example, the information exemplified in FIG. 3b tells that the product type A is the one to be administered (“Product” value), that the total target of the day of product A is 300 grams (“Target” value), that the day in question 200 grams of product A have already been administered to the pet (“Today” value) and that nothing (zero value) is being weighed in that moment in the weighing part 21 (“Actual” value).

In another moment of time of the day, administration of the product (product type A, for example) is done to the pet, as represented in FIG. 3c. In this Figure it can be seen that the product type A is the one to be administered (“Product” value), and that the total target of the day of product A is 300 grams (“Target” value): the two upper values remain invariable with respect to those in FIG. 3b. Then, as 50 grams of product A are being weighed in that moment in the weighing part 21 (“Actual” value), the cumulative quantity of product A for the day passes now from 200 grams in FIGS. 3b to 250 grams in FIG. 3c (“Today” value). These two lower quantities (“Today” and “Actual”) vary and are therefore adjusted by the user of the device 20, such as (but not limited to) by using the buttons 23 and 24: button 23 (+) typically used to indicate the device 20 and the controller 10 to which it is functionally linked the food intake (positive); button 24 (−) typically used to indicate the food removal (negative), for example food not having been eaten by the pet and that we want to throw away from the pet's food container, or for example, if an error was made and a certain quantity of food needs to be removed.

As already described by referring to FIGS. 3b and 3c, the two upper information (“Product” and “Target”) remain fixed for the day, whereas the two lower information (“Today” and “Actual”) vary according to the quantity weighed and the cumulative quantity already given that day. For this reason, a particular (but non-limiting) execution of the device represents these two lower quantities (“Today” and “Actual”) in a different colour so the user can easily follow the daily program.

The method on which the system of the present disclosure is based is described in Document WO2014/031836 A1 belonging to the same applicant: the method is based on preserving lean body mass and preventing or minimizing loss of lean body mass during weight loss by animals, preventing a reduction in energy metabolism and maintaining a higher daily energy expenditure, during weight loss by animals, reducing the risk of regaining weight by animals after weight loss, and ameliorating undesirable animal behaviors associated with reduced caloric intake. This method comprises two feeding periods: a caloric reduction period and a caloric maintenance period. During the caloric maintenance period, animals are fed a diet containing calories that meet the animals' maintenance energy requirements. During the caloric reduction period, the animals are fed with a diet containing calories that do not meet the animals' maintenance energy requirements. It has been found that animals fed by intermittent feeding patterns will preserve and maintain lean body mass and maintain, a higher energy expenditure while losing same amount of weight as the animals fed low caloric diets continuously; in addition, the animal's behavior will be more desirable. Further, the animals will lose weight while preserving lean body mass, retaining energy metabolism and not regaining weight that has been lost as a result of reduced caloric intake. Additionally, the methods ameliorate undesirable animal behaviors associated with hunger that results from reduced caloric intake, e.g., begging for food, seeking food, binge eating, voracious eating, anxiety, aggression, depression, excessive vocalization, and the like.

While feeding for a the caloric reduction period and for a caloric maintenance period as described is often sufficient, the particular (but non-limiting) method requires that the feeding pattern is repeated for at least two cycles, for several cycles, for as long as the animal, benefits from, the feeding pattern (e.g., achieves a desired weight loss), or for the life of the animal. The caloric reduction period and the caloric maintenance period can be periods of the same duration or of a different duration. In certain non-limiting embodiments, the caloric reduction period and the caloric maintenance period are intermittent periods of the same duration.

Generally, the caloric reduction diet contains fewer calories than the caloric maintenance diet in any number of calories sufficient to maintain a higher energy expenditure during weight loss, preserve lean body mass during weight loss, or achieve any related or associated benefit described herein. In certain non-limiting embodiments, the caloric reduction diet contains from five percent (5%) to ninety percent (90%) fewer calories than the caloric maintenance diet, such as (but not limited to) from ten percent (10%) to seventy percent (70%), or from twenty percent (20%) to forty (40%). In certain embodiments, the caloric reduction diet contains fewer calories than the caloric maintenance diet in amounts of from about 5% to about 95% of the calories needed to meet the animal's daily maintenance requirement for energy, such as (but not limited to) from about 10% to about 80%, or from about 15% to about 60%. Any suitable amount is acceptable depending upon the desired results.

In certain non-limiting embodiments, the method of the present disclosure is useful for managing weight loss and related functions described herein for companion animals, particularly those whose diet is controlled by an owner or caregiver. In a particular (but non-limiting) embodiment, the animals are dogs and cats.

Moreover, the present disclosure further relates to composition kits 30 comprising certain products of certain types, in a given quantity each of them, to be administered to the pet in a certain total period of time and under certain time frequencies. Typically, the system of the present disclosure works with different kits 30, prepared specifically for a certain nutritional program that was established according to the invariable parameters 11 and the variable parameters 12 given to the controller 10. Therefore, there shall be a certain composition kit 30 for a specific nutritional program established. It is also possible, according to another embodiment of the present disclosure, that there is no pre-existing kit prepared comprising all the contents needed to follow a certain nutritional program but instead, that the user of the system will have certain different quantities of different products that he will properly administer to the pet at certain frequencies, as required by the nutritional program shown to the user in the screen 22.

It is also possible that, once a certain nutritional program 31 has ended or is about to end, a certain alert is sent to the device 20 so the user of the system can agree to command automatically another composition kit 30 that will be suitable for a new nutritional program 31, established after the variable parameter 12 (typically the new weight of the pet) has been updated. The ordering of the new composition kit 30 is made by the controller 10 (once the user has agreed by pressing an “ok” button or similar in the device 20 in order to agree with the new request); the controller being typically a smartphone, a router or a PC, the command or request of the new kit is done automatically in a web page or web site.

It is also possible that the composition kits 30 further comprise one or more weight loss agents to be administered in conjunction with the feeding products. Any weight loss agent that is compatible with the feeding patterns of the present disclosure can be used in the present disclosure, in various embodiments, the weight loss agent is one or more of carnitine, isoflavones, pyruvate, fish oil, DMA, EPA, fibers, calcium, resistant starch, medium chain triglycerides, green tea extract, phentemine, diethylpropion, orlistat, sibutramine, megestrol phenylpropanolamine, oxandrolone. oleoylethanolamide, probioties that promote weight loss, ephedra, conjugated imoleic acid, glucomannan, and the like.

Although the present disclosure has been described with reference to particular embodiments thereof, many modifications and alternations may be made by a person having ordinary skill in the art without departing from the scope of the present disclosure which is defined by the appended claims.

1. A pet weight management system comprising:
a controller receiving data from a pet, these data comprising at least one or more invariant parameters and one or more variable parameters, the controller establishing a nutritional program as a function of these data, the nutritional program comprising at least one or a plurality of feeding cycles, each feeding cycle comprising at least a caloric reduction period and at least a caloric maintenance period;
a nutrition management device operatively linked to the controller receiving the information on the nutritional program to follow, and dosing and/or dispensing via weighing or volume measurement; and
a composition kit comprising the required nutritional products in the required quantities needed to follow the established nutritional program.

2. The pet weight management system according to claim 1, wherein the caloric reduction period and the caloric maintenance period are intermittent periods of the same duration.

3. The pet weight management system according to claim 1, wherein the caloric reduction period contains from 5% to 90% fewer calories than the caloric maintenance period.

4. The pet weight management system according to claim 1, wherein the invariant parameters are at least one or a plurality of: breed, age, gender, activity level, behavior, and/or sterilization.

5. The pet weight management system according to claim 1, wherein the variable parameters are at least the pet's weight.

6. The pet weight management system according to claim 1, wherein the nutritional program comprises at least one or a plurality of the following data: type of nutritional product or composition, quantity of the nutritional product or composition, total duration and/or administration frequencies of the nutritional program.

7. A nutrition management device for use in the system of claim 1, the nutrition management device comprising:
a weighing device further configured to accommodate at least part of the nutritional products to be administered to the pet.

8. The nutrition management device according to claim 7, configured as a dosing device.

9. The nutrition management device according to claim 7, further comprising a wireless connection mode allowing its connection to the controller in the system.

10. The nutrition management device according to claim 7, configured to synchronize data automatically each time it is near the controller.

11. The nutrition management device according to claim 7, further comprising a screen where the information on the nutritional program to follow is graphically represented.

12. The nutrition management device according to claim 7, further comprising at least one or more operating buttons configured to adjust one or more parameters from the nutritional program.

13. A composition kit for use in the system of claim 1, wherein the composition kit is adapted to follow a certain nutritional program.

14. A composition kit for use in the system of claim 1, the composition kit comprising at least one or more nutritional products that can be administered to follow one or more nutritional programs.

15. The composition kit according to claim 13, further comprising at least one or a plurality of weight loss agents taken from the list of: carnitine, isoflavones, pyruvate, fish oil, DMA, EPA, fibers, calcium, resistant starch, medium chain triglycerides, green tea extract, phentemine, diethylpropion, orlistat, sibutramine, megestrol phenylpropanolamine, oxandrolone, oleoylethanolamide, probioties that promote weight loss, ephedra, conjugated imoleic acid, and glucomannan.

16. A method for promoting weight loss in a pet, using a system according to claim 1, the method comprising:
establishing a certain nutritional program as a function of one or more invariant parameters and one or more variable parameters, the nutritional program comprising at least one or a plurality of feeding cycles, each feeding cycle comprising at least a caloric reduction period and at least a caloric maintenance period;
updating the nutritional program established in a nutrition management device; and
following the nutritional program through the device, operatively linked to the controller.

17. The method for promoting weight loss in a pet according to claim 16, wherein a new updated nutritional program is established each time at least one of the variable parameters changes.

18. The method for promoting weight loss in a pet according to claim 16, wherein the caloric reduction period and the caloric maintenance period are intermittent periods of the same duration.

19. The composition kit according to claim 14, further comprising at least one or a plurality of weight loss agents taken from the list of: carnitine, isoflavones, pyruvate, fish oil, DMA, EPA, fibers, calcium, resistant starch, medium chain triglycerides, green tea extract, phentemine, diethylpropion, orlistat, sibutramine, megestrol phenylpropanolamine, oxandrolone, oleoylethanolamide, probioties that promote weight loss, ephedra, conjugated imoleic acid, and glucomannan.
